(19) United States
(12) Patent Application Publication
(10) Pub. No.: US 2008/0021287 A1
(43) Pub. Date: Jan. 24, 2008

(54) System and method for adaptively adjusting patient data collection in an automated patient management environment

(76) Inventors: Matthias D. Woellenstein, Overijse (BE); Howard D. Simms, Shoreview, MN (US)

Correspondence Address: CASCADIA INTELLECTUAL PROPERTY, 500 UNION STREET, STE.1005, SEATTLE, WA, 98101, US

(21) Appl. No.: 11/475,642

(22) Filed: Jun. 26, 2006

(51) Int. Cl. A61B 5/00

(52) U.S. Cl. 600/300

(57) ABSTRACT

A system and method for adaptively adjusting patient data collection in an automated patient management environment is presented. A patient is monitored through continual remote patient management. Physiological measures are collected from the patient on a substantially regular basis. The collected physiological measures are analyzed to evaluate patient status based on an assessment to recognize a trend in status quo, progression, regression, onset, or absence of a health condition affecting the patient. An actionable change in the patient is identified. The collection of the physiological measures is dynamically adjusted in response to the actionable change.

### FIELD OF THE INVENTION

The invention relates in general to automated patient management and, specifically, to a system and method for adaptively adjusting patient data collection in an automated patient management environment.

### BACKGROUND OF THE INVENTION R

emote patient management enables a clinician, such as a physician, nurse, or other healthcare provider, to follow patient well-being through homecare medical devices that can collect and forward patient data without requiring the presence or assistance of medical personnel. Remote patient management can be provided over a data communications network, such as the Internet. One or more medical devices per patient are remotely interconnected with a centralized server via dedicated patient management devices, such as repeaters, installed in patients' homes. The patient management devices supplement traditional programmers that interrogate patient medical devices in-clinic. This infrastructure allows patient well-being to be continually monitored and centrally analyzed by professional healthcare staff without the costs of office visits.

Remote patient management can enable early identification of changes to patient well-being, including rapid onset of acute conditions or gradual onset of chronic conditions, although the changes detected through monitored physiological measures could also be the result of disease or other adverse health condition, as well as attributable to innocuous factors, such as improper diet or fatigue. Frequently, the first indication that a change in patient well-being might require medical attention is the subjective qualitative feelings of the patient, who may be suffering headaches, lethargy, or other physical discomfort.

Remote patient management carries the inherent potential of data overload due to the possible volume of information collectible for an entire patient population, particularly where frequent collection, reporting, and analysis are performed. While routinely collecting patient data facilitates monitoring and evaluating patient wellness and status, over-monitoring patients whose health conditions are stable and unchanging can unnecessarily consume resources, including network communication bandwidth, processing cycles, and storage capacity. While increased monitoring may be advisable to adaptively accommodate those patients presenting with actual or perceived concerns to their physical well-being, conventional approaches to remote patient monitoring nevertheless adopt static patient data collection models.

U.S. Pat. No. 6,168,563, to Brown, discloses a system and method that enables a healthcare provider to monitor and manage a health condition of a patient. A clearinghouse computer communicates with the patient through a data management unit, which interactively monitors the patient's health condition by asking questions and receiving answers that are supplied back to the clearinghouse computer. Patient information may also be supplied by physiological monitoring devices, such as a blood glucose monitor or peak-flow meter. Healthcare professionals can access the patient information through the clearinghouse computer, which can process, analyze, print, and display the data. However, the fixed periodicity of data collection is defined at the discretion of health professionals and is not automatically adjusted as health conditions change.

U.S. Pat. No. 6,416,471, to Kumar et al. (“Kumar”), discloses a portable remote patient telemonitoring device. A disposable sensor band with electro-patches detects and transmits vital signs data to a signal transfer unit, which can be either be worn or positioned nearby the patient. The base station receives data transmissions from the signal transfer unit for transferring the collected data to a remote monitoring station. Indications are provided to a patient from a base station when threshold violations occur. However, the frequency of data collection is fixed and changes to data collection must be specified manually.

U.S. Pat. No. 6,024,699, to Surwit et al. (“Surwit”), discloses a central data processing system configured to communicate with and receive data from patient monitoring systems, which may implement medical dosage algorithms to generate dosage recommendations. Blood from a pricked finger may be read on a chemically treated strip for review at the central data processing system. Modifications to medicine dosages, the medicine dosage algorithms, patient fixed or contingent self-monitoring schedules, and other treatment information are communicated. However, the system is reliant on the patient to notice and adjust to changes in self-monitoring schedules to affect the frequency of data collection.

U.S. Pat. No. 6,827,670, to Stark et al. (“Stark”), discloses a system for medical performance management. A monitoring device, such as a personal orthopedic restraining device, monitors patient actions relative to a biological manipulation protocol being performed on a patient with an orthopedic injury under treatment according to a coordinated, monitored recovery scheme. A portable, preferably handheld, computer records data from the monitoring device and provides the data to a centralized computer for analysis. Overall, the system functions as a goal-directed, closed loop monitoring system; however, the collection of monitored data is fixed per the treatment protocol currently prescribed to the patient.

Therefore, there is a need for adaptively tailoring the collection of physiological measures responsive to changes in the well-being of patients. Preferably, such an approach would automatically increase or decrease data collection metrics relative to temporal, volumetric, and compositional sampling needs in response to objective and subjective patient inputs.

### SUMMARY OF THE INVENTION

A system and method includes changing the manner in which patient data is collected by a centralized server, patient management devices, or medical devices, including internal and external medical therapy devices and medical sensors. The patient data can include both qualitative and quantitative physiological measures that are measured directly from or are indirectly provided by a patient under treatment. Clinician-specified, automated, and patient-specified criteria are implemented as triggers that effect a change in patient data collection. The change can effect temporal, volumetric, and compositional patient data collection metrics on at least one of the devices and one or more changes can be in effect at any given time.

One embodiment provides a system and method for adaptively adjusting patient data collection in an automated patient management environment. A patient is monitored through continual remote patient management. Physiological measures are collected from the patient on a substantially regular basis. The collected physiological measures are analyzed to evaluate patient status based on an assessment to recognize a trend in status quo, progression, regression, onset, or absence of a health condition affecting the patient. An actionable change in the patient is identified. The collection of the physiological measures is dynamically adjusted in response to the actionable change.

Still other embodiments of the present invention will become readily apparent to those skilled in the art from the following detailed description, wherein are described embodiments of the invention by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF THE DRAWINGS

FIG. 1 is a functional block diagram showing, by way of example, an automated patient management environment.

FIG. 2 is a graph diagram showing, by way of example, patient status and data collection periodicities as functions of time.

FIG. 3 is a data flow diagram showing, by way of example, patient data input sources in the environment of FIG. 1.

FIG. 4 is a data flow diagram showing, by way of example, patient data collection adjustment triggers in the environment of FIG. 1.

FIG. 5 is a data flow diagram showing, by way of example, patient data collection metrics in the environment of FIG. 1.

FIG. 6 is a Venn diagram showing dynamic physiological measure collection adjustment in the environment of FIG. 1.

FIG. 7 is a process flow diagram showing adaptively adjusting patient data collection in an automated patient management environment, in accordance with one embodiment.

FIG. 8 is a block diagram showing a system for adaptively adjusting patient data collection in an automated patient management environment, in accordance with one embodiment.

### DETAILED DESCRIPTION

Automated Patient Management Environment

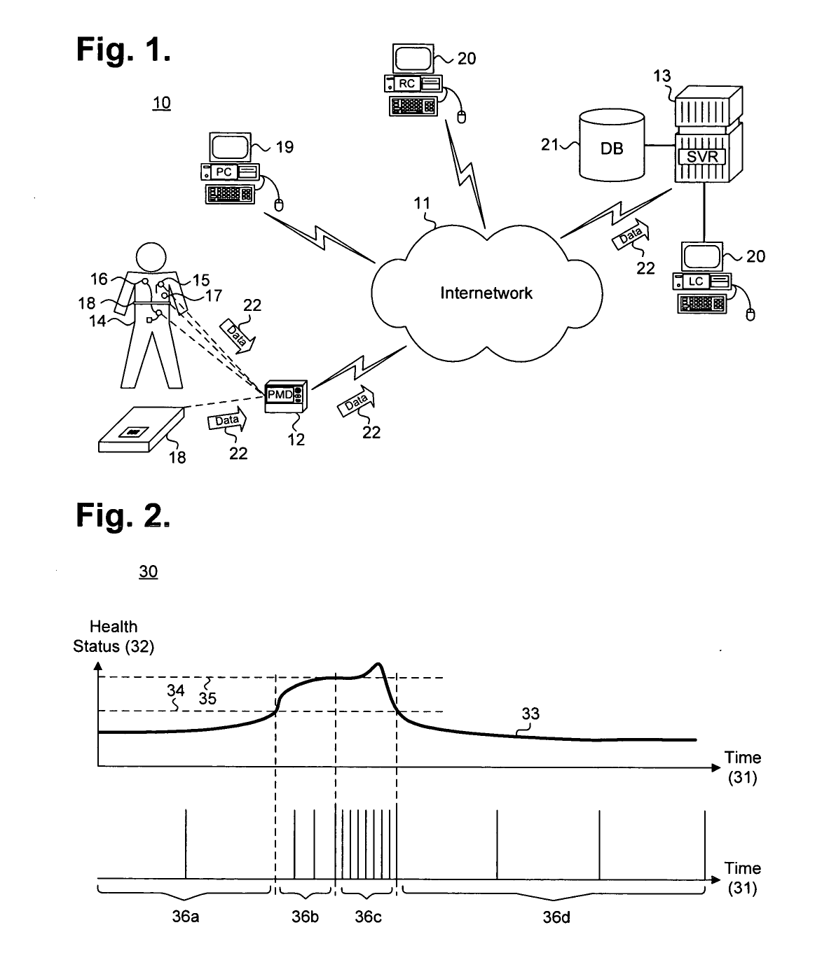

Automated patient management encompasses a range of activities, including remote patient management and automatic diagnosis of patient health, such as described in commonly-assigned U.S. Patent application Pub. No. US2004/0103001, published May 27, 2004, pending, the disclosure of which is incorporated by reference. Such activities can be performed proximal to a patient, such as in the patient's home or office, centrally through a centralized server, such from a hospital, clinic or physician's office, or through a remote workstation, such as a secure wireless mobile computing device. FIG. 1 is a functional block diagram showing, by way of example, an automated patient management environment 10. In one embodiment, a patient 14 is proximal to one or more patient monitoring or communications devices, such as a patient management device 12, which are interconnected remotely to a centralized server 13 over an internetwork 11, such as the Internet, or through a public telephone exchange (not shown), such as a conventional or mobile telephone network. Other patient monitoring or communications devices are possible. In addition, the internetwork 11 can provide both conventional wired and wireless interconnectivity. In one embodiment, the internetwork 11 is based on the Transmission Control Protocol/Internet Protocol (TCP/IP) network communication specification, although other types or combination of networking implementations are possible. Similarly, other network topologies and arrangements are possible.

Each patient management device 12 is uniquely assigned to a patient under treatment 14 to provide a localized and network-accessible interface to one or more medical devices 15-18, either through direct means, such as wired connectivity, or through indirect means, such induction or as selective radio frequency or wireless telemetry based on, for example, “strong” Bluetooth or IEEE 802.11 wireless fidelity “WiFi” and “WiMax” interfacing standards. Other configurations and combinations of patient data source interfacing are possible. Medical therapy devices include implantable medical devices (IMDs) 15, such as pacemakers, implantable cardiac defibrillators (ICDs), drug pumps, and neuro-stimulators, and external medical devices (EMDs) 16, such as automatic external defibrillators (AEDs). Medical sensors include implantable sensors 17, such as implantable heart and respiratory monitors and implantable diagnostic multi-sensor non-therapeutic devices, and external sensors 18, such as Holter monitors, weight scales, and blood pressure cuffs. Other types of medical therapy, medical sensing, and measuring devices, both implantable and external, are possible.

Patient data includes physiological measures, which can be quantitative or qualitative, parametric data regarding the status and operational characteristics of the patient data source itself, and environmental parameters, such as the temperature or time of day. The medical devices 15-18 collect and forward the patient data 22 either as a primary or supplemental function. The medical devices 15-18 include, by way of example, implantable and external medical therapy devices that deliver or provide therapy to the patient 14, implantable and external medical sensors that sense physiological data in relation to the patient 14, and measurement devices that measure environmental parameters and other data occurring independent of the patient 14. Other types of patient data are possible. Each medical device 15-18 can generate one or more types of patient data and can incorporate one or more components for delivering therapy, sensing physiological data, measuring environmental parameters, or a combination of functionality.

Ordinarily, patient data 22 is collected by the medical devices 15-18 for forwarding to a patient management device 12, which can analyze and, in turn, also forward the patient data 22 to the centralized server 13. By default, each medical device 15-18, patient management device 12, and the centralized server 13 collect the patient data 22 at a fixed rate, which, in one embodiment, can vary by proximity to the patient under treatment 14. For instance, medical devices 15-18, which are immediately proximal to a patient, generally collect patient data 22 most frequently, such as on a per episode or scheduled basis, whereas the patient management device 12 and centralized server 13 respectively collect patient data 22 on a daily and weekly bases. Other data collection periodicities are possible. Upon sensing or receiving notice that patient status has or might have changed, the parameters defining patient data collection metrics can be dynamically adjusted in response to the change or other factors, as further described below beginning with reference to FIG. 2 et seq. Clinician-specified, automated, and patient-specified criteria define a set of triggers that can cause the temporal, volumetric, or compositional metrics for patient data collection to increase or decrease based on the type of change in patient well-being sensed or on other factors. The patient data collection metrics can be further adjusted, as required, as patient well-being improves or deteriorates and normal patient data collection can be resumed upon patient recovery, clinician directive, or other direction.

In a further embodiment, data values can be directly entered by a patient 14. For example, answers to health questions could be input into a patient system 19, such as a personal computer with user interfacing means, such as a keyboard, display, microphone, and speaker. Such patient-provided data values could also be collected as patient information. In one embodiment, the medical devices 15-18 collect the quantitative objective physiological measures on a substantially continuous or scheduled basis and also record the occurrence of events, such as therapy or irregular readings. In a further embodiment, the patient management device 12, patient system 19, or similar device record or communicate qualitative subjective quality of life (QOL) measures that reflect the personal impression of physical well-being perceived by the patient 14 at a particular time. Other types of patient data collection, periodicity and storage are possible in a further embodiment, the collected patient data can also be accessed and analyzed by one or more clients 20, either locally-configured or remotely-interconnected over the internetwork 11. The clients 20 can be used, for example, by clinicians to securely access stored patient data 22 assembled in a database 21 and to select and prioritize patients for health care provisioning, such as respectively described in commonly-assigned U.S. patent application Ser. No. 11/121,593, filed May 3, 2005, pending, and U.S. patent application Ser. No. 11/121,594, filed May 3, 2005, pending, the disclosures of which are incorporated by reference. Although described herein with reference to physicians or clinicians, the entire discussion applies equally to organizations, including hospitals, clinics, and laboratories, and other individuals or interests, such as researchers, scientists, universities, and governmental agencies, seeking access to the patient data.

In a further embodiment, patient data 22 is safeguarded against unauthorized disclosure to third parties, including during collection, assembly, evaluation, transmission, and storage, to protect patient privacy and comply with recently enacted medical information privacy laws, such as the Health Insurance Portability and Accountability Act (HIPAA) and the European Privacy Directive. At a minimum, patient health information that identifies a particular individual with health- and medical-related information is treated as protectable, although other types of sensitive information in addition to or in lieu of specific patient health information could also be protectable.

Preferably, the server 13 is a server-grade computing platform configured as a uni-, multi- or distributed processing system, and the patient systems 19 and clients 20 are general-purpose computing workstations, such as a personal desktop or notebook computer. In addition, the patient management device 12, server 13, patient systems 19, and clients 20 are programmable computing devices that respectively execute software programs and include components conventionally found in computing device, such as, for example, a central processing unit (CPU), memory, network interface, persistent storage, and various components for interconnecting these components.

Patient Status and Data Collection Periodicity

The collection of patient data is performed on a fixed schedule subject to dynamic adjustment due to changes in the status of the patient under treatment. FIG. 2 is a graph diagram 30 showing, by way of example, patient status and data collection periodicities 36a-d as functions of time 31. The x-axis represents time 31 and the y-axis represents the health status 32 of the patient under treatment 14.

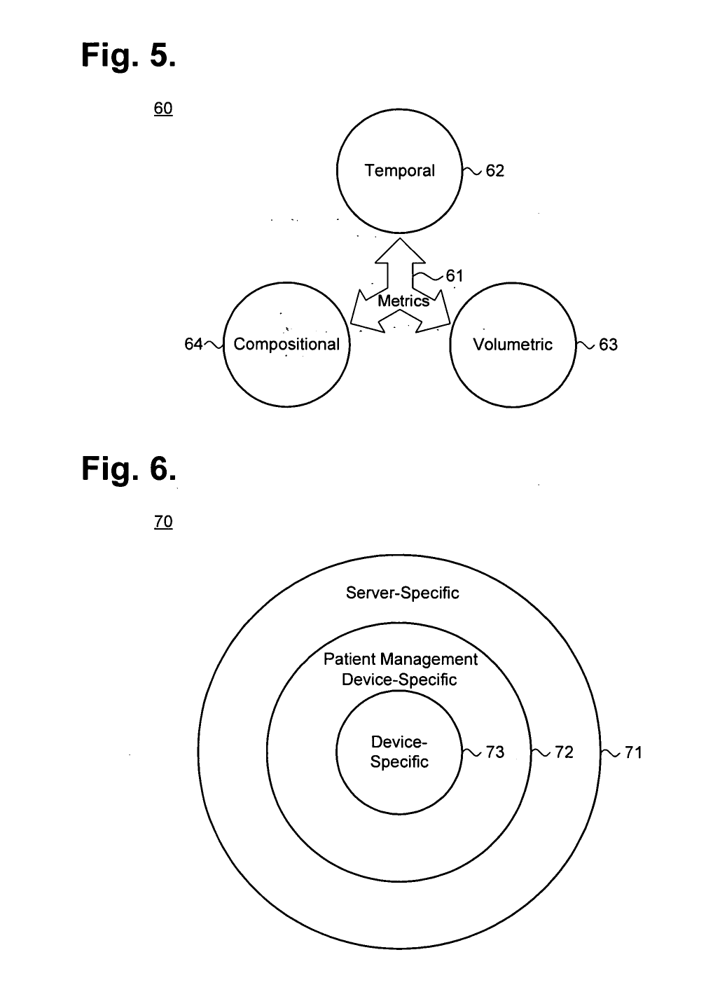

Patient health status 32 can be tracked as a function 33 over time 31. The function 33 can be based on objective, quantitative physiological measures or subjective qualitative patient inputs, taken individually or as an aggregate or combination and can respectively be compared to upper and lower thresholds 34, 35 that can trigger dynamic adjustments to patient data collection. The frequency of patient data collection can be tracked as a set of periodicities 36a-d, which each signify time periods for patient data collection at different fixed rates. Patient data collection periodicities can vary between infinity, which signifies no patient data collection, to zero, which signifies real time patient data collection. Transitions to different patient data collection periodicities can be triggered by thresholds 34, 35 or other criteria, as further described below with reference to FIG. 4. For example, a patient data collection periodicity changing from one sample per day 36a to one sample every four hours 36b could be triggered when the patient health status 32 exceeds a lower threshold 34. Similarly, the patient data collection periodicity could transition to one sample every thirty minutes 36c when the patient health status 32 exceeds an upper threshold 35. Normal patient data collection periodicity of one sample per day 36d could resume when the patient health status 32 drops below the lower threshold 34. Other types of changes to the patient data collection metrics in addition to sampling rate could be used, either individually or in combination along with one or more criteria for each particular change or group of changes, as further described below with reference to FIG. 5. In addition, the dynamic adjustments to patient data collection could occur with respect to one or more triggers while the normal functioning of the devices affected by the changes continues to operate and regularly collect patient data. As well, changes to multiple patient data collection metrics could also apply.

Patient Data Input Sources

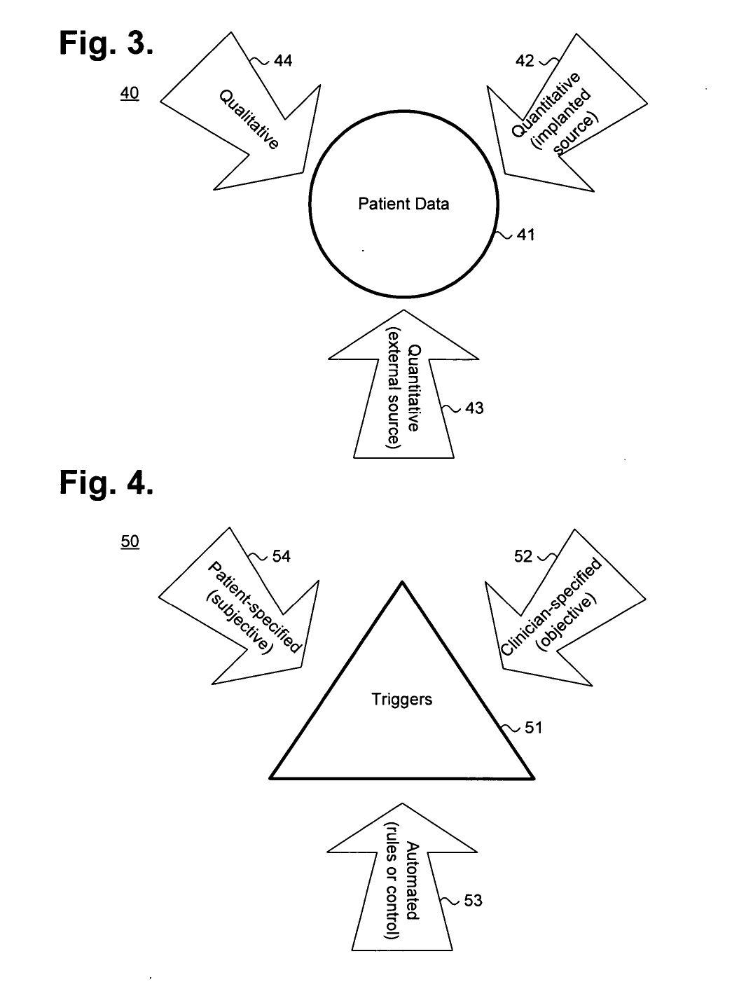

Patient data, in the form of physiological measures, can originate from direct and indirect sources. FIG. 3 is a data flow diagram 40 showing, by way of example, patient data input sources 42-44 in the environment 10 of FIG. 1. The composition of patient data 41 depends upon the patient data input sources 42-44 that contribute to the patient data 41. Generally, medical devices 15-18 are limited to collecting either objective quantitative or subjective qualitative patient data directly from the patient. In contrast, a patient medical device 12 can collect patient data 41 that includes quantitative measures from implanted sources 42 and external sources 43, as well as qualitative data 41, which could be obtained by presenting queries to probe patients' subjective perception of physical well-being. The centralized server 13 can also collect patient data 41 that includes quantitative physiological measures from implanted and external sources 42, 43 and qualitative physiological measures 44 for an entire patient population, subpopulation, or group. Thus, the characteristics of the patient data 41 are dependent upon the collection point. Other patient data input sources are possible.

Patient Data Collection Adjustment Triggers

Dynamic changes to the patient data collection can be triggered by quantitative and qualitative different criteria directly or indirectly affecting or influencing patient status. FIG. 4 is a data flow diagram 50 showing, by way of example, patient data collection adjustment triggers in the environment 10 of FIG. 1. One or more adjustments to patient data collection can be triggered either individually by or as an aggregate or combination of each criteria. Patient data collection adjustment triggers 51 include clinician-specified criteria 52, automated criteria 53, and patient-specified criteria 54. Other automated triggers are possible.

Clinician-specified criteria 52 can include quantitative or qualitative triggers based on instructions received from a clinician. Quantitative triggers generally set objective thresholds or other automated limits or boundary conditions. Qualitative triggers generally respond to subjective patient instructions. Both types of triggers adjust the patient data collection performed over one or more metrics. For example, a clinician could set an automated timer to perform patient data collection at the same time each day to observe a patient's reactions to a new treatment protocol. As a further example, a patient feeling palpitations who has also previously suffered a single sudden cardiac death episode and a myocardial infarction could seek care from her physician. To aid in diagnosing the patient, the physician could give the patient a magnet to trigger her IMD to record an ECG whenever she began to feel palpitations. The patient would be instructed to notify the physician, who could then retrieve and analyze the ECG data.

Automated criteria 53 can include quantitative triggers based on rules or control that are programmed into a device. Generally, such rules and control can be parameterized to allow clinicians to customize and fine-tune device behavior, but immutable rules and control are also possible. For example, a patient presenting with a progressively decreasing heart rate, such as a decrease from 70 beats per minute to 60 beats per minute, could trigger an automated criteria 53 to record and upload a rhythm strip on a daily basis to allow the patient to be followed for indications of an adverse health condition.

Finally, patient-specified criteria 54 can include qualitative triggers based on subjective perceptions of patients of their physical well-being. Patient-specified criteria 54 are qualitative in nature and could be implemented either as a trigger 51 through a rule or control or by patient-initiated action. Qualitative physiological measures are generally provided in response to queries that probe a patient concerning their perceived physical well-being and certain types of patient responses can be implemented to trigger an adjustment, often temporary, to patient data collection. For example, a patient reporting shortness of breath could require temporarily increased data collection, at least while the complaint of shortness of breath continues.

Each of the triggers 51 can work either individually or in conjunction with other triggers 51 and can result in one or more changes to patient data collection for specific, aggregated, or combined types of physiological measures.

Patient Data Collection Metrics

The type of change made to patient data collection depends upon the metric affected. FIG. 5 is a data flow diagram 60 showing, by way of example, patient data collection metrics in the environment 10 of FIG. 1. The type of patient data collection metric 61 affected will depend in part on the trigger 51 causing the change. Temporal metrics 62 affect the frequency and duration of patient data sampling. Volumetric metrics 63 affect the number of samples and the amount or size of a sample taken at one particular sampling. Compositional metrics 64 affect the kinds of physiological measures collected, including quantitative and qualitative data. One or more of the metrics 61 can be modified in response to a change in patient data collection. In addition, the changes to the metrics 61 can be affected either separately or in aggregate or combination with other patient data collection changes, as well as in addition to or in lieu of regular on-going patient data collection. Other types of patient data collection metrics are possible.

Dynamic Physiological Measure Collection Adjustment

Data collection changes can be effected at one or more levels of the remote patient management infrastructure. FIG. 6 is a Venn diagram 70 showing dynamic physiological measure collection adjustment in the environment 10 of FIG. 1. The most general form of patient data collection adjustment occurs at a server-specific level 71. Such changes occur for a patient group or population to modify the parameters controlling patient data collection for the centralized server 13, patient management devices 12, or medical devices 15-18.

Changes to patient data collection that are particular to an individual patient 14 are most appropriately effected at a patient management device-specific level 72. Such changes affect only the patient management device 12 dedicated to a patient and could also affect one or more medical devices 15-18.

Generally, medical devices 15-18 operate in an event- or episode-based manner, but could be configured to effect changes to patient data collection on a device-specific level 73. Such changes require the medical device 15-18 to augment on-going therapy or sensing with additional monitoring and data collection based on triggered criteria. Changes at a device-specific level 73 would not ordinarily affect the data collection performed by the associated patient management device 12 or centralized server 13, but could indirectly trigger those devices to also modify the patient data collection performed to accommodate the medical device 15-18.

Method

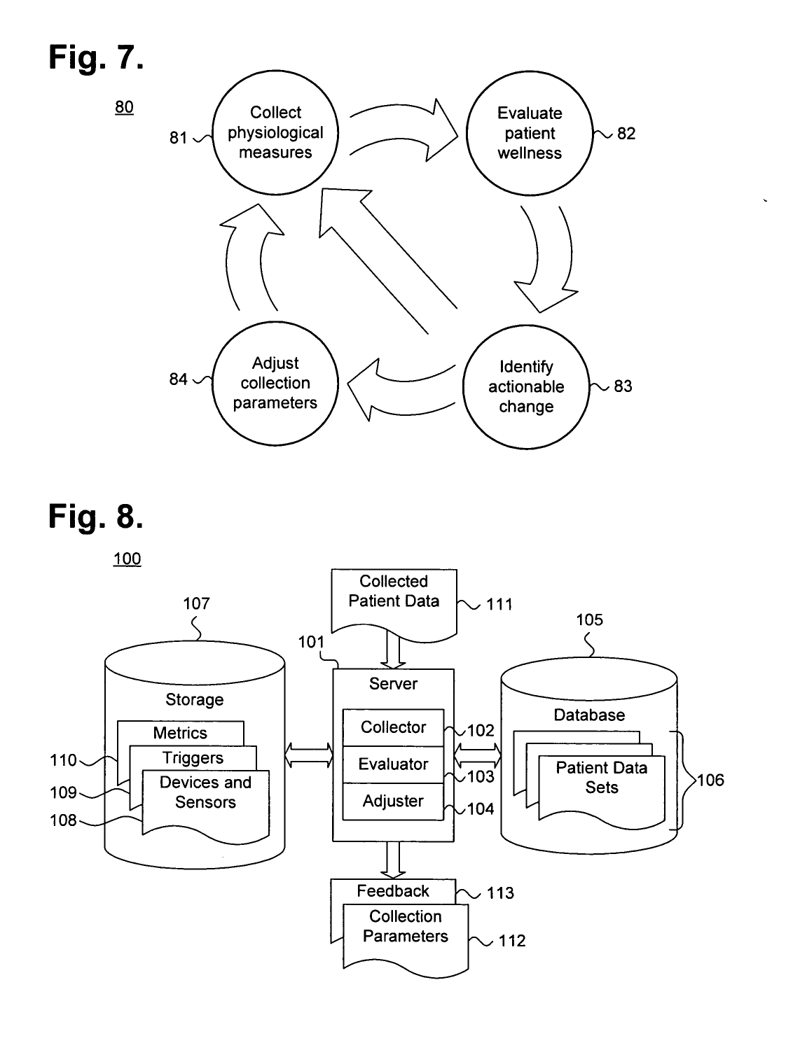

Patient data is collected at all levels beginning with medical devices 15-18, patient management devices 12, and the centralized server 13. FIG. 7 is a process flow diagram 80 showing adaptively adjusting patient data collection in an automated patient management environment 10, in accordance with one embodiment. Dynamic changes to patient data collection follow a continual cycle independent of the level of the infrastructure affected. During each cycle, physiological measures are collected (operation 81), which can include both qualitative and quantitative data. The physiological measures could be collected either as part of on-going monitoring or sensing, to diagnose a particular medical concern, or for another purpose.

Patient status is then evaluated (operation 82). Patient status is assessed by quantitatively and qualitatively analyzing the patient data holistically to recognize trends indicating a health condition or the absence of a health condition potentially affecting the patient. The trends can include a progression, regression, onset, or absence of a medical concern, as well as a status quo or unchanging condition. Other types of trends could be recognized. In a further embodiment, the patient data is analyzed for non-trending aberrations that may also indicate a health condition or the absence of a health condition of medical concern.

Any actionable changes in the well-being of the patient are identified (operation 83). An actionable change would be the result of a triggering condition based on clinician-specified, automated, or patient-specified criteria, such as described above with reference to FIG. 4. Collection of physiological measures (operation 81) resumes if no actionable changes are identified. Otherwise, the patient data collection parameters are adjusted (operation 84) to change one or more patient data collection metrics, such as described above with reference to FIG. 5, after which physiological measure collection continues (operation 81). Other operations either in addition to or in lieu of the foregoing operations are possible.

Server

Changes to patient data collection can be implemented on the centralized server, patient management devices, and medical devices. The most general form of patient data collection changes affects the centralized server and patient data collection changes to patient management devices and medical devices affect a subset of the centralized server's functionality. FIG. 8 is a block diagram 100 showing a system for adaptively adjusting patient data collection in an automated patient management environment 10, in accordance with one embodiment. A server 101 executes a sequence of programmed process steps, such as described above with reference to FIG. 7, implemented, for instance, on a programmed digital computer.

The server 101 includes storage 107 and database 105 and can be configured to coordinate the displaying of patient data for multiple patients between a plurality of patient systems 19, clients 20, and other compatible computing systems. Other server functions are possible.

The server 101 includes a collector 102, evaluator 103, and adjuster 104. The collector 102 maintains a list of devices and sensors 108 for all medical devices 15-18 and patient management devices 12. The collector 102 receives collected patient data 111, which is stored as patient data sets 106 in the database 105. The collector 102 collects patient data on both an on-going basis and as modified by adjustments to patient data collection.

The evaluator 103 evaluates the collected patient data 111 against triggers 109, which implement clinician-specified, automated, and patient-specified criteria. The collected patient data 111 is evaluated to recognize trends in patient well-being that could indicate a potential health condition or absence of a health condition of possible medical concern affecting the patient. The trends include patient status quo, progression, regression, onset, or an absence of a health concern. In addition, the evaluator 103 can provide feedback 113 to indicate the type of trigger 109 triggered and the underlying patient data.

The adjuster 104 effects changes to patient data collection by adjusting the metrics 110 associated with the devices subject to the change in patient data collection. Collection parameters 112 are sent to the affected devices to modify the programmed rules and control or to request the collection of patient data directly. Other types of server operations are possible.

While the invention has been particularly shown and described as referenced to the embodiments thereof, those skilled in the art will understand that the foregoing and other changes in form and detail may be made therein without departing from the spirit and scope of the invention.

What is claimed is:

1. A system for adaptively adjusting patient data collection in an automated patient management environment, comprising:
a device to monitor a patient through continual remote patient management, comprising:
a collector to collect physiological measures from the patient on a substantially regular basis; and
an analyzer to analyze the collected physiological measures to evaluate patient status based on an assessment to recognize a trend in status quo, progression, regression, onset, or absence of a health condition affecting the patient; and
an adjuster to identify an actionable change in the patient, and to dynamically adjust the collection of the physiological measures in response to the actionable change.

2. A system according to claim 1, wherein the physiological measures comprise at least one of objective quantitative and subjective qualitative measures.

3. A system according to claim 1, wherein the patient is queried to probe the subjective qualitative measures.

4. A system according to claim 1, wherein the collection of the physiological measures is modified through at least one of temporal, volumetric, and compositional changes.

5. A system according to claim 1, wherein the collection of the physiological measures is modified, comprising at least one of increasing the collection of the physiological measures upon identifying a trend indicating a progression or onset of an adverse health condition, and decreasing the collection of the physiological measures upon identifying a trend indicating a regression or absence of an adverse health condition.

6. A system according to claim 5, wherein the collection of the physiological measures is maintained upon identifying a trend indicating a status quo of an adverse health condition.

7. A system according to claim 1, further comprising:
at least one of clinician-specified, automated, and patient-specified criteria to define the actionable change.

8. A system according to claim 1, wherein the dynamic adjustment is effected on one of one-time, limited, scheduled, recurring, and real time bases.

9. A system according to claim 1, wherein the dynamic adjustment is effected on one or more of a centralized server, patient management device, and patient medical device or sensor.

10. A system according to claim 1, further comprising:
at least one of an implantable medical device, an external medical device, an implantable sensor, and an external sensor to collect the physiological measures.

11. A method for adaptively adjusting patient data collection in an automated patient management environment, comprising:
monitoring a patient through continual remote patient management, comprising:
collecting physiological measures from the patient on a substantially regular basis; and
analyzing the collected physiological measures to evaluate patient status based on an assessment to recognize a trend in status quo, progression, regression, onset, or absence of a health condition affecting the patient;
identifying an actionable change in the patient; and
dynamically adjusting the collection of the physiological measures in response to the actionable change.

12. A method according to claim 11, wherein the physiological measures comprise at least one of objective quantitative and subjective qualitative measures.

13. A method according to claim 11, further comprising:
querying the patient to probe the subjective qualitative measures.

14. A method according to claim 11, further comprising:
modifying the collection of the physiological measures through at least one of temporal, volumetric, and compositional changes.

15. A method according to claim 11, further comprising:
modifying the collection of the physiological measures, comprising at least one of:
increasing the collection of the physiological measures upon identifying a trend indicating a progression or onset of an adverse health condition; and
decreasing the collection of the physiological measures upon identifying a trend indicating a regression or absence of an adverse health condition.

16. A method according to claim 15, further comprising:
maintaining the collection of the physiological measures upon identifying a trend indicating a status quo of an adverse health condition.

17. A method according to claim 11, further comprising:
defining the actionable change through at least one of clinician-specified, automated, and patient-specified criteria.

18. A method according to claim 11, further comprising:
effecting the dynamic adjustment on one of one-time, limited, scheduled, recurring, and real time bases.

19. A method according to claim 11, further comprising:
effecting the dynamic adjustment on one or more of a centralized server, patient management device, and patient medical device or sensor.

20. A method according to claim 11, further comprising:
collecting the physiological measures via at least one of an implantable medical device, an external medical device, an implantable sensor, and an external sensor.

21. A computer-readable storage medium holding code for performing the method according to claim 11.

22. An apparatus adaptively adjusting patient data collection in an automated patient management environment, comprising:
means for monitoring a patient through continual remote patient management, comprising:
means for collecting physiological measures from the patient on a substantially regular basis; and
means for analyzing the collected physiological measures to evaluate patient status based on an assessment to recognize a trend in status quo, progression, regression, onset, or absence of a health condition affecting the patient;
means for identifying an actionable change in the patient; and
means for dynamically adjusting the collection of the physiological measures in response to the actionable change.
